(19) United States
(12) Patent Application Publication
(10) Pub. No.: US 2023/0165674 A1
(43) Pub. Date: Jun. 1, 2023

(54) ACHROMATIC IOL WITH MULTIPLE LAYERS OF DIFFRACTIVE OPTICS

(71) Applicant: Alcon Inc., Fribourg (CH)

(72) Inventors: Shinwook Lee, Arlington, TX (US); Xin Hong, Fort Worth, TX (US); Myoung-Taek Choi, Arlington, TX (US); Zhiguang Xu, Fort Worth, TX (US)

(21) Appl. No.: 18/055,656

(22) Filed: Nov. 15, 2022

Related U.S. Application Data
(60) Provisional application No. 63/284,318, filed on Nov. 30, 2021.

(51) Int. Cl. A61F 2/16

(52) U.S. Cl. CPC A61F 2/1635; A61F 2/1648; A61F 2/1694; A61F 2002/1682; A61F 2002/16901; A61F 2210/0014; A61F 2250/0003

(57) ABSTRACT

A multi-layer intraocular lens (IOL) includes a lens body, including an anterior diffractive optics layer, comprising a first biocompatible material, and a posterior diffractive optics layer, comprising a second biocompatible material that is different from the first biocompatible material. The anterior diffractive optics layer and the posterior diffractive optics layer are sealed in a peripheral non-optic portion of the lens body with a gap between the anterior diffractive optics layer and the posterior diffractive optics layer.

### PRIORITY CLAIM

This application claims the benefit of priority of U.S. Provisional Patent Application Ser. No. 63/284,318 titled “ACHROMATIC IOL WITH MULTIPLE LAYERS OF DIFFRACTIVE OPTICS,” filed on Nov. 30, 2021, whose inventors are Myoung-Taek Choi, Xin Hong, Shinwook Lee and Zhiguang Xu, which is hereby incorporated by reference in its entirety as though fully and completely set forth herein.

### BACKGROUND

A state-of-the art intraocular lens (IOL) that uses one layer of diffractive optics often provides high diffractive efficiency only at or near its design wavelength. More specifically, for a state-of-the art IOL, diffractive efficiency often decreases as wavelength of light deviates from the design wavelength. An existing hybrid IOL having a refractive surface and a diffractive surface can compensate for the wavelength dependency of focal length, also referred to as achromatization. However, the achromatization is limited due to the low diffraction efficiency for the broadband spectrum. Such low diffraction efficiency causes light leakage to undesirable orders of diffraction and thus reduces image quality.

Therefore, there is a need for IOLs that compensate for the variation of the diffraction efficiencies throughout the entire visible light spectrum.

### SUMMARY

Aspects of the present disclosure provide a multi-layer intraocular lens (IOL). The multi-layer IOL includes a lens body, including an anterior diffractive optics layer, comprising a first biocompatible material, and a posterior diffractive optics layer, comprising a second biocompatible material that is different from the first biocompatible material. The anterior diffractive optics layer and the posterior diffractive optics layer are sealed in a peripheral non-optic portion of the lens body with a gap between the anterior diffractive optics layer and the posterior diffractive optics layer.

Aspects of the present disclosure also provide a multi-layer intraocular lens (IOL). The multi-layer IOL includes a lens body, including an anterior diffractive optics layer, and a posterior diffractive optics layer, bonded to the anterior diffractive optics layer in a peripheral non-optic portion of the lens body. The lens body has diffractive efficiency of between 80% and 100% for the visible light spectrum.

Aspects of the present disclosure further provide a method for configuring multi-layer intraocular lens (IOL). The method includes computing a radial spacing and a step height of a first set of annular echelettes on a posterior surface of an anterior diffractive optics layer of an IOL and a radial spacing and a step height of a second set of annular echelettes on an anterior surface of a posterior diffractive optics layer of the IOL based on input parameters, and forming the IOL or causing the IOL to be formed based on the computed radial spacing and the computed step height of the first set of annular echelettes and the computed radial spacing and the computed step height of the second set of annular echelettes. The input parameters comprise a first refractive index of a first biocompatible material associated with the anterior diffractive optics layer and a second refractive index of a second biocompatible material associated with the posterior diffractive optics layer.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1A illustrates a top view of a multi-layer intraocular lens (IOL), according to certain embodiments.

FIG. 1B illustrates a side view of a lens body of the IOL of FIG. 1A, according to certain embodiments.

FIG. 1C illustrates a conventional single-layer IOL, according to certain embodiments.

FIG. 2 illustrates diffraction efficiency of a mono-focal multi-layer IOL and a mono-focal single-layer IOL, according to certain embodiments.

FIGS. 3 and 4 depict modulation transfer functions (MTFs) of a mono-focal multi-layer IOL and a mono-focal single-layer IOL, according to certain embodiments.

FIG. 5 depicts visual acuity of a mono-focal multi-layer IOL and a mono-focal single-layer IOL, according to certain embodiments.

FIGS. 6 and 7 depict MTFs of an enhanced depth of focus (EDOF) multi-layer IOL and an EDOF single layer IOL, according to certain embodiments.

FIG. 8 depicts visual acuity of an EDOF multi-layer IOL and an EDOF single layer IOL, according to certain embodiments.

FIGS. 9 and 10 depict MTFs of a tri-focal multi-layer IOL and a tri-focal single-layer IOL, according to certain embodiments.

FIG. 11 depicts visual acuity of a tri-focal multi-layer IOL and a tri-focal single-layer IOL, according to certain embodiments.

FIG. 12 depicts an example system for designing, configuring, and/or forming a multi-layer IOL, according to certain embodiments.

FIG. 13 depicts example operations for forming a multi-layer IOL, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The embodiments described herein provide multi-layer intraocular lenses (IOLs). A multi-layer IOL includes two or more layers of diffractive optics and can achieve both achromatization (i.e., reducing or eliminating the wavelength dependence of focal length) and enhancement of diffractive efficiency throughout the entire visible light spectrum, as compared to a conventional single-layer IOL. A multi-layer IOL can further enhance the modulation transfer function (MTF) and visual acuity as compared to a conventional single-layer IOL.

### Multi-Layer IOL

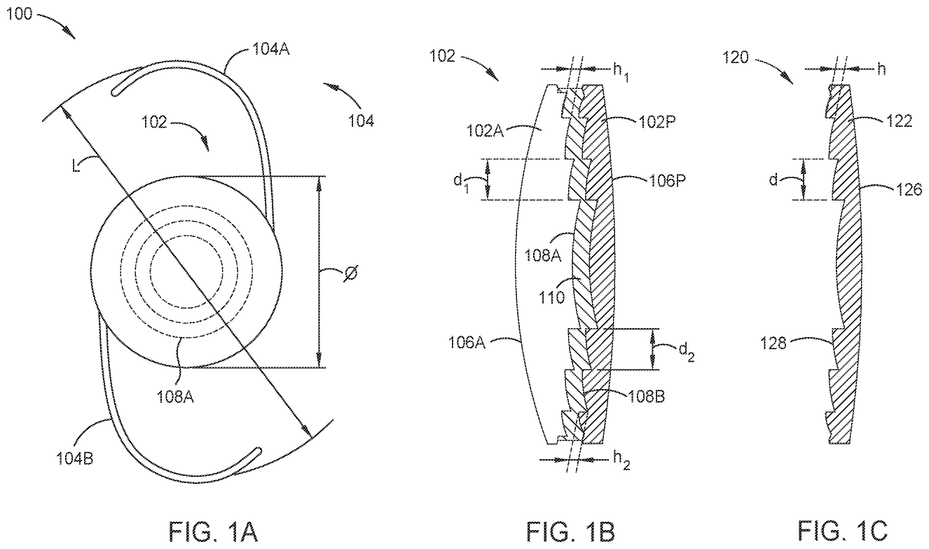

FIG. 1A illustrates a top view of a multi-layer intraocular lens (IOL) 100, according to certain embodiments. The multi-layer IOL 100 includes a lens body 102 and a haptic portion 104 that is coupled to a peripheral, non-optic portion of the lens body 102. FIG. 1B illustrates a side view of the lens body 102. It is noted that the shape and curvatures of the lens body 102 are shown for illustrative purposes only and that other shapes and curvatures are also within the scope of this disclosure. For example, the lens body 102 shown in FIG. 1A has a bi-convex shape. In other examples, the lens body 102 may have a plano-convex shape, a convexo-concave shape, or a plano-concave shape.

The lens body 102 has a diameter of between about 4.5 mm and about 7.5 mm, for example, about 6.0 mm. The lens body 102 includes an anterior diffractive optics layer 102A having an anterior outer surface 106A with a radius of curvature R₁, and a posterior diffractive optics layer 102P having a posterior outer surface 106P with a radius of curvature R₂. In certain embodiments, as shown in FIG. 1B, the multi-layer IOL 100 is a multi-focal IOL (with multiple focal points, e.g., bi-focal and tri-focal) having a first set of annular echelettes 108A on a posterior surface (i.e., a surface opposite to the anterior outer surface 106A) of the anterior diffractive optics layer 102A and a second set of annular echelettes 108B on an anterior surface (i.e., a surface opposite to the posterior outer surface 106P) of the posterior diffractive optics layer 102P.

The annular echelettes 108A, 108B each form concentric circular gratings. The annular echelettes 108A have a radial spacing d₁ between two adjacent annular echelettes and each have a step height h₁. The annular echelettes 108B have a radial spacing d₂ between two adjacent annular echelettes 108B and each have a step height h₂. The step height h₁ may be the same for all annular echelettes 108A or different for different annular echelettes 108A, and between about 1 μm and about 300 μm, for example, 35 μm.

The step height h₂ may be the same for all annular echelettes 108B or different for different annular echelettes 108B, and between about 1 μm and about 300 μm, for example, 41 μm. The radial spacing d₁ may be the same for all annular echelettes 108A or different for different annular echelettes 108A, and between about 10 μm and about 2000 μm, for example, 500 μm. The radial spacing d₂ of the annular echelettes 108B may coincide with the radial spacing d₁ of the annular echelettes 108A such that the annular echelettes 108A are proximate and opposed to the annular echelettes 108B.

Note that although in FIG. 1B, the first set of annular echelettes 108A is formed on a posterior surface of the anterior diffractive optics layer 102A and the second set of annular echelettes 108B is formed on the anterior surface of the posterior diffractive optics layer 102P, in certain other embodiments, not shown in FIG. 1B, the first set of annular echelettes 108A is formed on the anterior outer surface 106A and the second set of annular echelettes 108B is formed on the posterior outer surface 106P.

Further, note that, although multi-layer IOL 100 is a multi-focal IOL, in some other embodiments (not shown), the multi-layer IOL 100 is a mono-focal IOL (with one focal point) without annular echelettes on the outer surfaces (not shown). In some other embodiments, the multi-layer IOL 100 is an extended depth of focus (EDOF) IOL (with elongated focus) having annular echelettes on the posterior outer surface 106P.

The diffractive optics layers 102A and 102P may be bonded together to make a seal in a peripheral non-optic portion of the lens body 102, by chemical bonding, thermal bonding, UV bonding or other appropriate types of bonding, with a gap 110 between the diffractive optics layers 102A and 102P. Thickness of the gap 110 may be between about 1 μm and about 1000 μm, for example, 20 μm. The gap 110 may be filled with air or water-like fluid similar to aqueous humor, for instance, balanced salt solution (BSS). In certain embodiments, the annular echelettes 108A, 108B are fabricated on the diffractive optics layers 102A, 102P, respectively, prior to the bonding of the diffractive optics layers 102A and 102P. In certain other embodiments, the annular echelettes 108A, 108B are fabricated by laser writing or other appropriate techniques, subsequent to the bonding of the diffractive optics layers 102A and 102P having no annular echelettes.

The diffractive optics layers 102A, 102P may be each fabricated of a transparent, flexible, biocompatible material, such as a silicone polymeric material, acrylic polymeric material, hydrogel polymeric material. Young's Modulus, indicating stiffness and flexibility, of the two materials of which the diffractive optics layers 102A, 102P are fabricated may be between about 10 and about 300 MPa at dry 23° C. and between about 0.3 and about 100 MPa at hydrated 35° C., which are suitable for a multi-layer IOL 100 to be implemented inside a human eye. For example, the Young's Modulus of a first IOL material may be between about 140 MPa and 150 MPa at dry 18° C., between about 56 MPa and 66 MPa at dry 23° C., and between about 2.3 MPa and 2.5 MPa at hydrated 35° C. The Young's modulus of a second IOL material may be between about 130 MPa and 140 MPa MPa at dry 18° C., between about 60 MPa and 70 MPa at dry 23° C., and between about 2.0 MPa and 2.2 MPa at hydrated 35° C. Swelling factors (i.e., indication of expansion or shrinkage of the materials when immersed in the eye) of the two materials of which the diffractive optics layers 102A, 102P are fabricated may be similar, between 0% and 15%, for example, about 0.5% and 0.6%, having a difference less than about 5%, to ensure the seal between the diffractive optics layers 102A and 102P.

The diffractive optics layers 102A, 102P may have refractive indices n_{d1} and n_{d2}, and different Abbe numbers ν_{d1} and ν_{d2}, respectively. In certain embodiments, a difference between refractive indices n_{d1} and n_{d2} is between about 0 and 0.8. In some embodiments, Abbe numbers ν_{d1} and ν_{d2} may be between 25 and 50. In some embodiments, a difference between Abbe numbers ν_{d1} and ν_{d2} is between 5 and 60.0.

The anterior outer surface 106A of the anterior diffractive optics layer 102A and/or the posterior outer surface 106P of the posterior diffractive optics layer 102P may be fabricated of a biocompatible material (e.g., polymethyl methacrylate (PMMA), stiffer than the material of the remaining portions of the diffractive optics layers 102A and 102P.

The haptic portion 104 includes radially-extending struts (also referred to as “haptics”) 104A and 104B. The haptics 104A and 104B may be fabricated of a biocompatible material, such as PMMA. The haptics 104A and 104B are coupled (e.g., glued or welded) to the peripheral portion of the lens body 102 or molded along with a portion of the lens body 102, and thus extend outwardly from the lens body 102 to engage the perimeter wall of the capsular sac of the eye to maintain the lens body 102 in a desired position in the eye. The haptics 104A and 104B typically have radial-outward ends that define arcuate terminal portions. The terminal portions of the haptics 104A and 104B may be separated by a length L of between about 6 mm and about 22 mm, for example, about 13 mm. The haptics 104A and 104B have a particular length so that the terminal portions create a slight engagement pressure when in contact with the equatorial region of the capsular sac after being implanted. While FIG. 1A illustrates one example configuration of the haptics 104A and 104B, any plate haptics or other types of haptics can be used.

### Achromatization and Diffraction Efficiency Enhancement

As discussed below in detail, a conventional single-layer IOL inevitably exhibits wavelength dependence of diffractive efficiency. Thus, the diffractive efficiency decreases as the wavelength differs from the design wavelength at which the diffractive efficiency is optimized. Furthermore, the diffractive efficiency, in principle, cannot reach 100% at wavelengths that are different from the design wavelength. The multi-layer IOL 100, according to certain embodiments described herein, can achieve simultaneously the achromatization (i.e., reducing or eliminating the wavelength dependence of focal length) and high diffraction efficiency of close to 100%, for example, between 80% and 100%, at any wavelength over the entire visible wavelength, or at least over a larger wavelength range than just the design wavelength, by adjusting parameters related to the diffractive optics layers 102A and 102P described above, such as the step heights h₁, h₂ of the annular echelettes 108A, 1088, given the refractive indices of the diffractive optics layers 102A and 102P. In addition, the radial spacings d₁, d₂ of the annular echelettes 108A, 108B, together with the step heights h₁, h₂ are adjusted to optimize performance of the multi-layer IOL 100, which can be measured in through-focus modulation transfer function (MTF), also referred to simply as MTF, visual acuity, and aberration. The radii of curvature R₁, R₂ of the diffractive optics layers 102A and 102P are determined according to a desired lens base power.

For the purposes of comparison with the multi-layer IOL 100, FIG. 1C depicts a conventional single-layer IOL 120 having a single diffractive optics layer 122. As shown in FIG. 1C, the single-layer IOL 120 is a multi-focal IOL having annular echelettes 128 on an anterior surface of the diffractive optics layer 122. The annular echelettes 128 have a radial spacing d between two adjacent annular echelettes and each have a step height h. In other cases, the single-layer IOL 120 may be a mono-focal IOL without annular echelettes (not shown). In yet other cases, the single-layer IOL 120 is an extended depth of focus (EDOF) IOL (with elongated focus) having annular echelettes (not shown) on a posterior outer surface 126.

For a single-layer IOL, such as the single-layer IOL 120, having a refractive index n(λ) at a wavelength λ, the first order diffraction efficiency η_{S}(λ) at the wavelength can be calculated with a scaler diffraction theory known in the art, as

$${η}_{S} ( λ ) = {sinc}^{2} ( \frac{{Φ}_{S} ( λ )}{2 π} - 1 ) ,$$

where sinc(x) is the sinc function,

$$sinc ( x ) := \frac{sin ( x )}{x} ,$$

and when the medium surrounding the IOL is air (i.e., n=1), Φ_{S}(λ) is the phase function defined as

$${Φ}_{S} ( λ ) = \frac{2 {πλ}_{0}}{λ} \frac{n ( λ ) - 1}{n ( {λ}_{0} ) - 1}$$

with the design wavelength λ₀. The step height h may be chosen to optimize the first order diffraction efficiency η_{S}(λ₀) at the design wavelength λ₀. Since the phase function Φ_{S}(λ) depends on the wavelength λ, the diffraction efficiency η_{S}(λ) varies as the wavelength varies λ.

Further, the diffraction efficiency η_{S}(λ) can reach 100% only when the phase function Φ_{S}(λ) equals 2π (i.e., the argument of the sinc function is zero). This condition is equivalently

$$n ( λ ) = \frac{λ}{{λ}_{0}} ( n ( {λ}_{0} ) - 1 ) + 1.$$

However, all known materials have a refractive index n(λ) that monotonically decreases as the wavelength λ increases, and thus the condition above cannot be not fulfilled at the wavelength λ, different from the design wavelength λ₀. Consequently, the diffraction efficiency η_{S}(λ) cannot reach 100% at the wavelength λ, different from the design wavelength λ₀.

For a multi-layer IOL, such as the multi-layer IOL 100, the first order diffraction efficiency η_{M}(λ) can be similarly calculated with the scaler diffraction theory, as

$${η}_{M} ( λ ) = {sinc}^{2} ( \frac{{Φ}_{M} ( λ )}{2 π} - 1 ) ,$$

where Φ_{M}(λ) is the phase function Φ_{M}(λ) defined as

$${Φ}_{M} ( λ ) = \frac{2 π {h}_{1}}{λ} ( {n}_{1} ( λ ) - 1 ) - \frac{2 π {h}_{2}}{λ} ( {n}_{2} ( λ ) - 1 )$$

where n₁(λ) and n₂(λ) are the refractive indices of the diffractive optics layers 102A and 102P, respectively, and the refractive index of the gap 110 (e.g., air) is assumed to be 1. Since the two terms in the phase function Φ_{M}(λ) have opposite signs, the dependency of the phase function Φ_{M}(λ) on the wavelength λ can be reduced as compared to the phase function Φ_{S}(λ) for the single-layer IOL 120, or eliminated, by appropriately adjusting the step heights h₁, h₂, given the refractive indices n₁(λ) and n₂(λ). Thus, high diffraction efficiency throughout the entire visible light spectrum can be achieved with the multi-layer IOL 100.

Further, the diffraction efficiency η_{M}(λ) can reach 100% when the phase function Φ_{M}(λ) equals 2π. This condition is equivalently

h₁(n₁(λ)−1)−h₂(n₂(λ)−1)=λ,

which can be fulfilled by appropriately adjusting the step heights h₁, h₂ at least at two different wavelengths λₐ, and λ_{b}, as

$${h}_{1} = \frac{{λ}_{a} ( {n}_{2} ( {λ}_{b} ) - 1 - {λ}_{b} ( {n}_{2} ( {λ}_{a} ) - 1 )}{( {n}_{1} ( {λ}_{a} ) - 1 ) ( {n}_{2} ( {λ}_{b} ) - 1 ) - ( {n}_{1} ( {λ}_{b} ) - 1 ) ( {n}_{2} ( {λ}_{a} ) - 1 )} ,$$

$${h}_{2} = \frac{{λ}_{a} ( {n}_{1} ( {λ}_{b} ) - 1 - {λ}_{b} ( {n}_{1} ( {λ}_{a} ) - 1 )}{( {n}_{1} ( {λ}_{a} ) - 1 ) ( {n}_{2} ( {λ}_{b} ) - 1 ) - ( {n}_{1} ( {λ}_{b} ) - 1 ) ( {n}_{2} ( {λ}_{a} ) - 1 )} ,$$

if the denominator is different from zero. The denominator is non-zero for real materials, if the two materials of which the diffractive optics layers 102A, 102P are fabricated are different.

It should be noted that these conditions for the step heights h₁, h₂ are tightly related to the Abbe numbers

$${v}_{d 1} = \frac{{n}_{1} ( {λ}_{D} ) - 1}{{n}_{1} ( {λ}_{F} ) - {n}_{1} ( {λ}_{C} )} , {v}_{d 2} = \frac{{n}_{2} ( {λ}_{D} ) - 1}{{n}_{2} ( {λ}_{F} ) - {n}_{2} ( {λ}_{C} )}$$

of the diffractive optics layers 102A and 102P, defined using the refractive indices n₁(λ), n₂(λ) at three different wavelengths λ_{F}=486.1 nm (blue Fraunhofer F line from hydrogen), λ_{D}=589.2 nm (orange Fraunhofer D line from sodium), and λ_{C}=656.3 nm (red Fraunhofer C line from hydrogen).

In general, the step heights h₁, h₂ can be smaller when a difference between the Abbe numbers ν₁, ν₂ is larger. For instance, the step heights are h₁=35.8 μm, h₂=40.5 μm for a combination of Material A and Material B (ν_{d1}=39.5, ν_{d2}=52.8). The step heights are h₁=317.0 μm, h₂=312.2 μm for a combination of Material A and Material C (ν_{d1}=39.5, ν_{d2}=37.3).

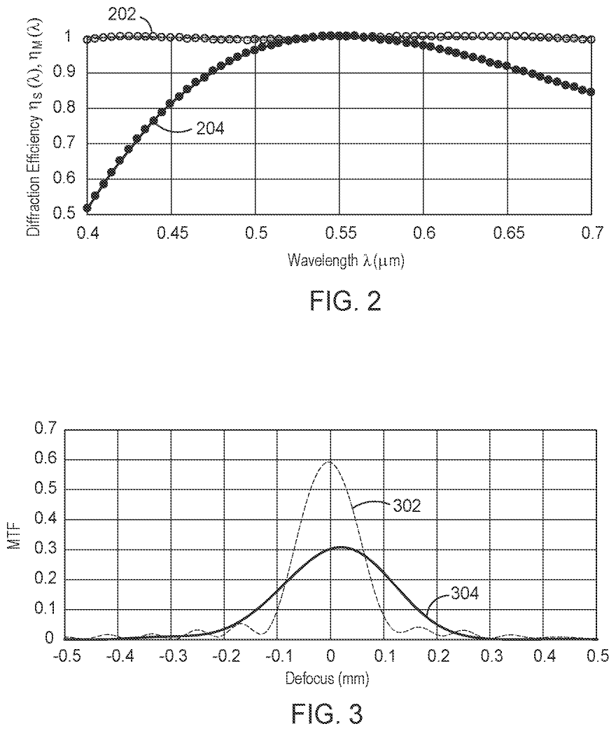

FIGS. 2-11 illustrate three different examples of the difference between the optical performance of various types of multi-layer and single layer IOLs.

### Example 1

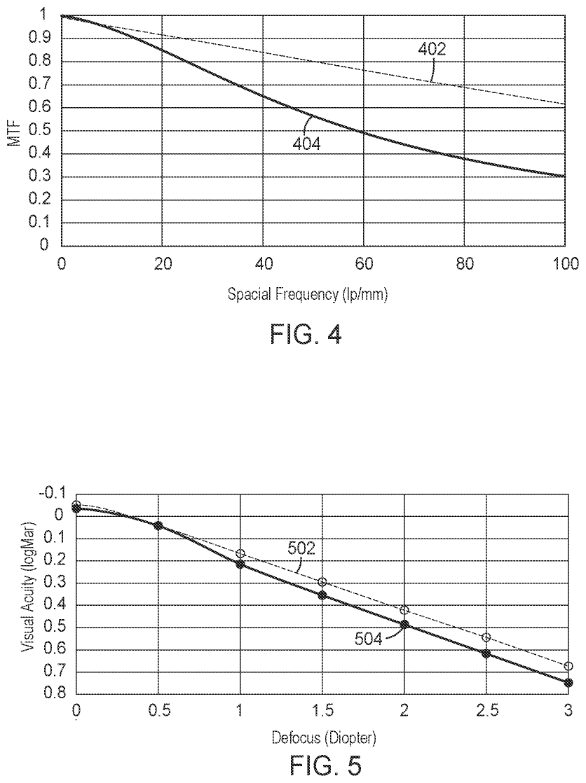

FIG. 2 depicts the diffraction efficiency, FIGS. 3 and 4 depict the MTFs, and FIG. 5 depicts the visual acuity of an example mono-focal multi-layer IOL, but without annular echelettes at outer surfaces, according to certain embodiments, in comparison to an example mono-focal single-layer IOL, such as the single-layer IOL 120 at a design wavelength λ₀ of 0.55 μm. In the example mono-focal multi-layer IOL, the anterior diffractive optics layer is fabricated of Material A with Abbe number ν_{d1} of 39.5 and the posterior diffractive optics layer is fabricated of Material B with Abbe number ν_{d2} of 52.8. In the comparison example mono-focal single-layer IOL, the diffractive optics layer is fabricated of Material A.

In FIG. 2, it can be seen that the mono-focal multi-layer IOL provides high diffraction efficiency 202 of between about 98% and about 100% for the entire visible light spectrum of wavelength λ of between about 0.4 μm and about 0.7 μm. However, the mono-focal single-layer IOL is designed such that its diffraction efficiency 204 is 100% at the design wavelength λ₀ of 0.55 μm, but the diffraction efficiency 204 decays rapidly as the wavelength λ deviates from the design wavelength λ₀.

In FIG. 3, the MTF mapping was generated by evaluating the MTF at different focus planes at 50 lp/mm (line pairs per millimeter) spatial resolution (also referred to as “spatial frequency”) using a 3-mm (photopic) aperture to determine a depth of focus (also referred to as “defocus”) for the IOLs. In FIG. 3, it can be seen the MTF 302 for the mono-focal multi-layer IOL has a narrower peak near the focal point (i.e., at zero defocus) than the MTF 304 for the mono-focal single-layer IOL. Thus, the mono-focal multi-layer IOL has higher focus than the mono-focal single-layer IOL. In FIG. 4, it can also be seen the mono-focal multi-layer IOL has enhanced MTF 402 as compared to the MTF 404 for the mono-focal single-layer IOL for various spatial frequencies.

In FIG. 5, simulated results 502, 504 for visual acuity of the mono-focal multi-layer IOL and the mono-focal single-layer IOL, respectively, are shown in terms of LogMAR (logarithm of the minimum angle of resolution) scores. The simulated result 502 of visual acuity of the mono-focal multi-layer IOL shows enhancement at a far distance (0 Diopter), an intermediate distance (1.5 Diopter), and a near distance (2.5 Diopter), as compared to a simulated result 504 of visual acuity of the mono-focal single-layer IOL.

### Example 2

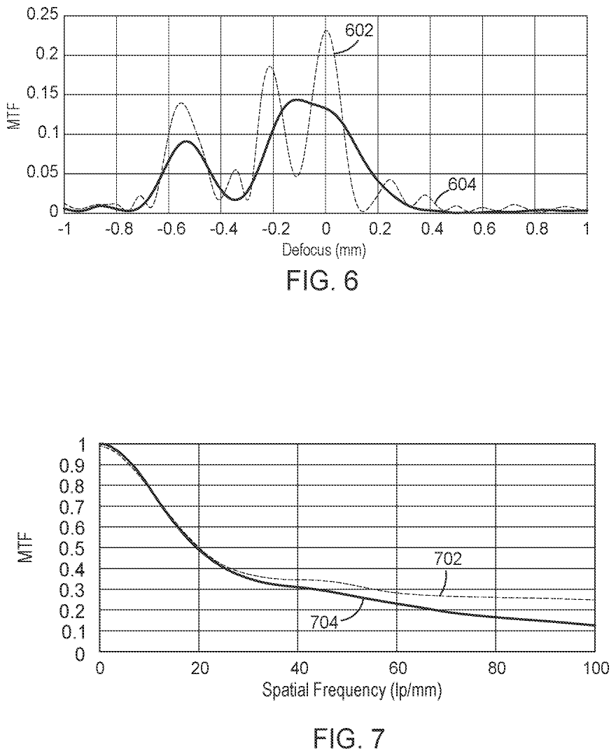

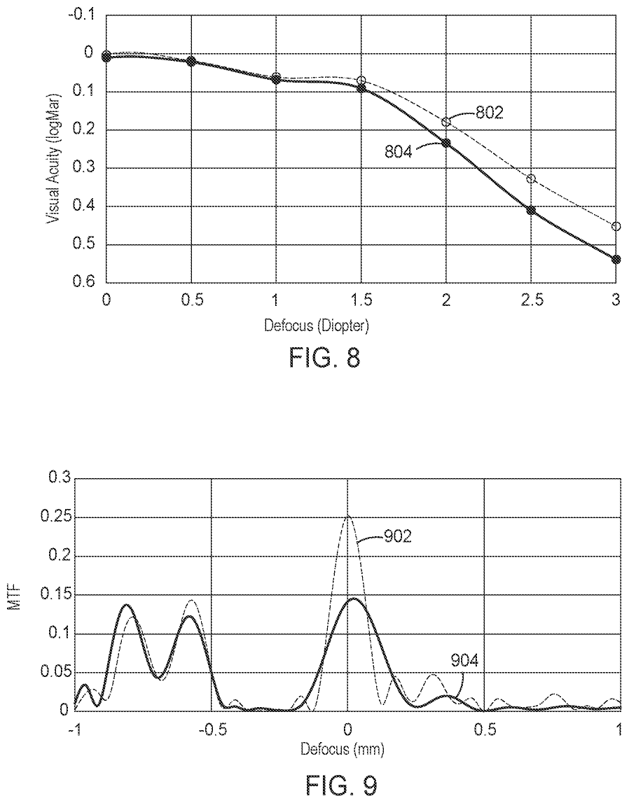

FIGS. 6 and 7 depict the MTFs and FIG. 8 depicts the visual acuity of an example EDOF multi-layer IOL, according to certain embodiments, in comparison to an example EDOF single layer IOL, such as the single-layer IOL 120.

In FIG. 6, the MTF mapping was generated by evaluating the MTF at different focus planes at 100 lp/mm (line pairs per millimeter) spatial frequency using a 3-mm (photopic) aperture to determine a depth of focus (also referred to as “defocus”) for the IOLs. In FIG. 6, it can be seen the MTF 602 for the EDOF multi-layer IOL has a narrower peak near the focal point (i.e., at zero defocus) than the MTF 604 for the EDOF single-layer IOL. Thus, the EDOF multi-layer IOL has higher focus than the EDOF single-layer IOL. In FIG. 7, it can also be seen the EDOF multi-layer IOL has enhanced MTF 702 at a far distance (0 Diopter) as compared to the MTF 704 for the EDOF single-layer IOL for various spatial frequencies.

In FIG. 8, simulated results 802, 804 for visual acuity of the EDOF multi-layer IOL and the EDOF single-layer IOL, respectively, are shown in terms of LogMAR. The simulated result 802 of visual acuity of the EDOF multi-layer IOL shows enhancement at a far distance (0 Diopter), an intermediate distance (1.5 Diopter), and a near distance (2.5 Diopter), as compared to a simulated result 804 of visual acuity of the EDOF single-layer IOL.

### Example 3

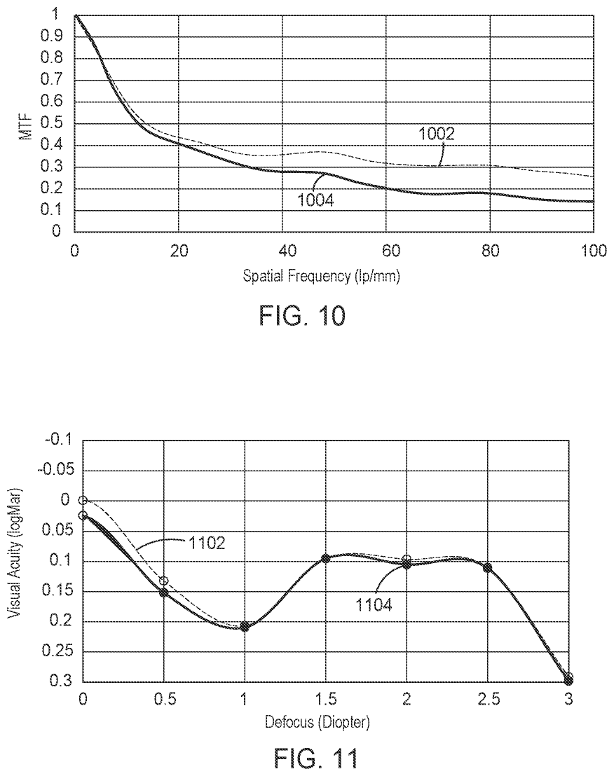

FIGS. 9 and 10 depict MTFs and FIG. 11 depicts visual acuity of an example tri-focal multi-layer IOL, according to certain embodiments, in comparison to an example tri-focal single-layer IOL, such as the single-layer IOL 120.

In FIG. 9, the MTF mapping was generated by evaluating the MTF at different focus planes at 100 lp/mm (line pairs per millimeter) spatial frequency using a 3-mm (photopic) aperture to determine a depth of focus (also referred to as “defocus”) for the IOLs. In FIG. 9, it can be seen the MTF 902 for the tri-focal multi-layer IOL has a narrower peak near the focal point (i.e., at zero defocus) than the MTF 904 for the tri-focal single-layer IOL. Thus, the tri-focal multi-layer IOL has higher focus than the tri-focal single-layer IOL. In FIG. 10, it can also be seen the tri-focal multi-layer IOL has enhanced MTF 1002 at a far distance (0 Diopter) as compared to the MTF 1004 for the tri-focal single-layer IOL for various spatial frequencies.

In FIG. 11, simulated results 1102, 1104 for visual acuity of the tri-focal multi-layer IOL and the tri-focal single-layer IOL, respectively, are shown in terms of LogMAR. The simulated result 1102 of visual acuity of the tri-focal multi-layer IOL shows enhancement at a far distance (0 Diopter), an intermediate distance (1.5 Diopter), and a near distance (2.5 Diopter), as compared to a simulated result 1104 of visual acuity of the tri-focal single-layer IOL.

### System for Designing Multi-Layer IOL

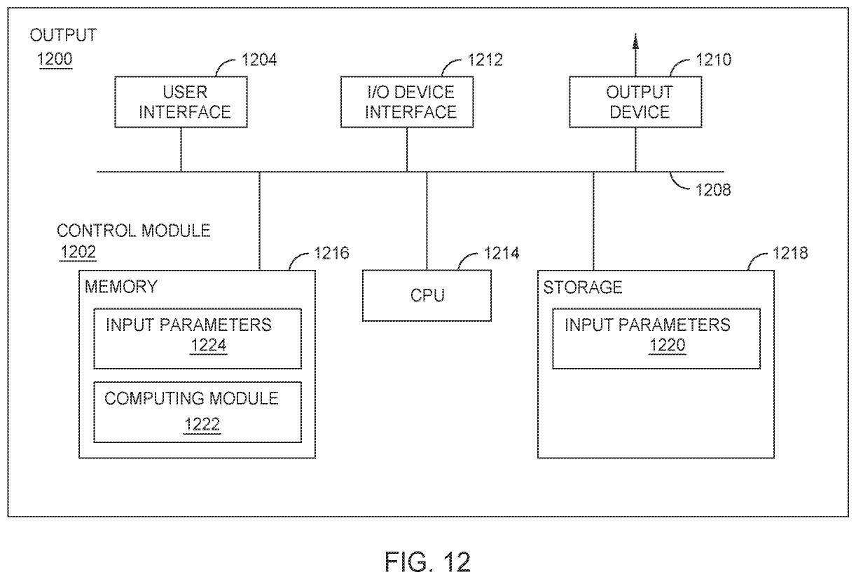

FIG. 12 depicts an exemplary system 1200 for designing, configuring, and/or forming a multi-layer IOL 100. As shown, the system 1200 includes, without limitation, a control module 1202, a user interface display 1204, an interconnect 1208, an output device 1210, and at least one I/O device interface 1212, which may allow for the connection of various I/O devices (e.g., keyboards, displays, mouse devices, pen input, etc.) to the system 1200.

The control module 1202 includes a central processing unit (CPU) 1214, a memory 1216, and a storage 1218. The CPU 1214 may retrieve and execute programming instructions stored in the memory 1216. Similarly, the CPU 1214 may retrieve and store application data residing in the memory 1216. The interconnect 1208 transmits programming instructions and application data, among CPU 1214, the I/O device interface 1212, the user interface display 1204, the memory 1216, the storage 1218, output device 1210, etc. The CPU 1214 can represent a single CPU, multiple CPUs, a single CPU having multiple processing cores, and the like. Additionally, in certain embodiments, the memory 1216 represents volatile memory, such as random access memory. Furthermore, in certain embodiments, the storage 1218 may be non-volatile memory, such as a disk drive, solid state drive, or a collection of storage devices distributed across multiple storage systems.

As shown, the storage 1218 includes input parameters 1220. The input parameters 1220 include a lens base power, asphericity, toricity, refractive indices n_{d1} and n_{d2} of the two materials of which the diffractive optics layers 102A, 102P are fabricated, and a design wavelength λ₀. The memory 1216 includes a computing module 1222 for computing control parameters such as the radial spacings d₁, d₂ and the step heights h₁, h₂ of the annular echelettes 108A, 108B. In addition, the memory 1216 includes input parameters 1224.

In certain embodiments, input parameters 1224 correspond to input parameters 1220 or at least a subset thereof. In such embodiments, during the computation of the control parameters, the input parameters 1224 are retrieved from the storage 1218 and executed in the memory 1216. In such an example, the computing module 1222 comprises executable instructions (e.g., including one or more of the formulas described herein) for computing the control parameters, based on the input parameters 1224. In certain other embodiments, input parameters 1224 correspond to parameters received from a user through user interface display 1204. In such embodiments, the computing module 1222 comprises executable instructions for computing the control parameters, based on information received from the user interface display 1204.

In certain embodiments, the computed control parameters, are output via the output device 1210 to a lens manufacturing system that is configured to receive the control parameters and form a lens accordingly. In certain other embodiments, the system 1200 itself is representative of at least a part of a lens manufacturing systems. In such embodiments, the control module 1202 then causes hardware components (not shown) of system 1200 to form the lens according to the control parameters. The details and operations of a lens manufacturing system are known to one of ordinary skill in the art and are omitted here for brevity.

### Method for Designing Multi-Layer IOL

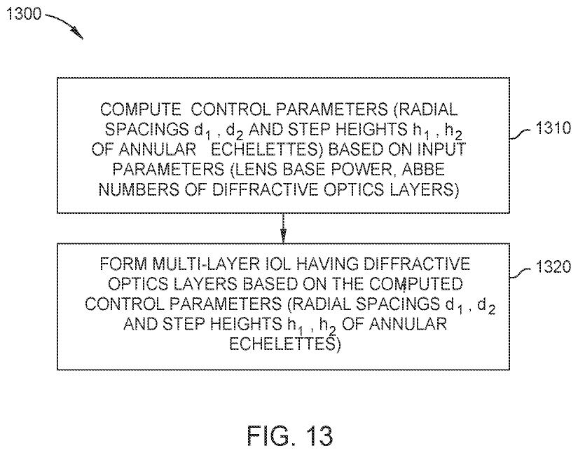

FIG. 13 depicts example operations 1300 for forming a multi-layer IOL. In some embodiments, the step 1310 of operations 1300 is performed by one system (e.g., the system 1200) while step 1320 is performed by a lens manufacturing system. In some other embodiments, both steps 1310 and 1320 are performed by a lens manufacturing system.

At step 1310, control parameters (e.g., the radial spacings d₁, d₂ and the step heights h₁, h₂ of the annular echelettes 108A, 108B) are computed based on input parameters (e.g., a lens base power, asphericity, toricity, refractive indices of the two materials of which the diffractive optics layers 102A, 102P are fabricated). The computations performed at step 1310 are based on one or more of the embodiments, including the formulas, described herein.

At step 1320, a multi-layer IOL (e.g., multi-layer IOL 100) having diffractive optics layers (e.g., diffractive optics layers 102A, 102P) based on the computed control parameters (e.g., the radial spacings d₁, d₂ and the step heights h₁, h₂ of the annular echelettes 108A, 108B) is formed, using appropriate methods, systems, and devices typically used for manufacturing lenses, as known to one of ordinary skill in the art.

The embodiments described herein provide multi-layer IOLs that can achieve both achromatization and high diffraction efficiency throughout the entire visible light spectrum, leading to significantly higher MTF and visual acuity, as compared to conventional single-layer IOLs. The enhancement of performance can be achieved with mono-focal IOLs, extended depth of focus (EDOF) multi-layer IOL, and tri-focal multi-layer IOLs.

While the foregoing is directed to embodiments of the present disclosure, other and further embodiments of the disclosure may be devised without departing from the basic scope thereof, and the scope thereof is determined by the claims that follow.

1. A multi-layer intraocular lens (IOL), comprising:
a lens body, comprising:
an anterior diffractive optics layer, comprising a first biocompatible material; and
a posterior diffractive optics layer, comprising a second biocompatible material that is different from the first biocompatible material,
wherein the anterior diffractive optics layer and the posterior diffractive optics layer are sealed in a peripheral non-optic portion of the lens body with a gap between the anterior diffractive optics layer and the posterior diffractive optics layer.

2. The multi-layer IOL of claim 1, wherein
the first biocompatible material has a swelling factor of between 0 and 15%,
the second biocompatible material has a swelling factor of between 0 and 15% and
the difference between the swelling factors of the first and the second biocompatible materials is less than 5%.

3. The multi-layer IOL of claim 1, wherein
the first biocompatible material has an Abbe number of between 5 and 60, and
the second biocompatible material has an Abbe number of between 5 and 60.

4. The multi-layer IOL of claim 3, wherein
the anterior diffractive optics layer includes a first set of annular echelettes on a posterior surface of the anterior diffractive optics layer, and
the posterior diffractive optics layer includes a second set of annular echelettes on an anterior surface of the posterior diffractive optics layer.

5. The multi-layer IOL of claim 4, wherein
a step height of the first set of annular echelettes is between 1 μm and 300 μm, and
a step height of the second set of annular echelettes is between 1 μm and 300 μm.

6. The multi-layer IOL of claim 4, wherein
a radial spacing of the first set of annular echelettes is between 10 μm and 2000 μm, and
a radial spacing of the second set of annular echelettes is between 10 μm and 2000 μm.

7. The multi-layer IOL of claim 1, wherein
the gap has a thickness of between 1 μm and 1000 μm.

8. The multi-layer IOL of claim 1, further comprising:
a haptic portion that is coupled to the lens body, the haptic portion comprising a third biocompatible material.

9. A multi-layer intraocular lens (IOL), comprising:
a lens body, comprising:
an anterior diffractive optics layer; and
a posterior diffractive optics layer, bonded to the anterior diffractive optics layer in a peripheral non-optic portion of the lens body,
wherein the lens body has diffractive efficiency of between 80% and 100% for the visible light spectrum.

10. The multi-layer IOL of claim 9, wherein
the anterior diffractive optics layer comprises a first biocompatible material; and
the posterior diffractive optics layer comprises a second biocompatible material that is different from the first biocompatible material.

11. The multi-layer IOL of claim 10, wherein
the first biocompatible material has a swelling factor of between 0 and 15%,
the second biocompatible material has a swelling factor of between 0 and 15% and
the difference between the swelling factors of the first and the second biocompatible materials is less than 5%.

12. The multi-layer IOL of claim 10, wherein
the first biocompatible material has an Abbe number of between 5 and 60, and
the second biocompatible material has an Abbe number of between 5 and 60.

13. The multi-layer IOL of claim 12, wherein
the anterior diffractive optics layer includes a first set of annular echelettes on a posterior surface of the anterior diffractive optics layer, and
the posterior diffractive optics layer includes a second set of annular echelettes on an anterior surface of the posterior diffractive optics layer.

14. The multi-layer IOL of claim 13, wherein
a step height of the first set of annular echelettes is between 1 μm and 300 μm, and
a step height of the second set of annular echelettes is between 1 μm and 300 μm.

15. The multi-layer IOL of claim 13, wherein
a radial spacing of the first set of annular echelettes is between 10 μm and 2000 μm, and
a radial spacing of the second set of annular echelettes is between 10 μm and 2000 μm.

16. A method for configuring a multi-layer intraocular lens (IOL), comprising:
computing a radial spacing and a step height of a first set of annular echelettes on a posterior surface of an anterior diffractive optics layer of an IOL and a radial spacing and a step height of a second set of annular echelettes on an anterior surface of a posterior diffractive optics layer of the IOL based on input parameters; and
forming the IOL or causing the IOL to be formed based on the computed radial spacing and the computed step height of the first set of annular echelettes and the computed radial spacing and the computed step height of the second set of annular echelettes,
wherein the input parameters comprise a first refractive index of a first biocompatible material associated with the anterior diffractive optics layer and a second refractive index of a second biocompatible material associated with the posterior diffractive optics layer.

17. The method of claim 16, wherein
the forming of the IOL or the causing of the IOL to be formed comprises:
bonding the anterior diffractive optics layer and the posterior diffractive optics layer in a peripheral non-optic portion of the anterior diffractive optics layer and the posterior diffractive optics layer; and
subsequent to the bonding, forming the first set of annular echelettes on the posterior surface of the anterior diffractive optics layer and the second set of annular echelettes on the anterior surface of the posterior diffractive optical layer.

18. The method of claim 16, wherein
the forming of the IOL or the causing of the IOL to be formed comprises:
forming the first set of annular echelettes on the posterior surface of the anterior diffractive optics layer and the second set of annular echelettes on the anterior surface of the posterior diffractive optical layer; and
subsequent to the forming, bonding the anterior diffractive optics layer and the posterior diffractive optics layer in a peripheral non-optic portion of the anterior diffractive optics layer and the posterior diffractive optics layer.

19. The method of claim 16, further comprising:
attaching a haptic portion to a peripheral non-optic portion of the anterior diffractive optics layer and the posterior diffractive optics layer.

20. The method of claim 16, wherein
the first biocompatible material has an Abbe number of between 25 and 50, and
the second biocompatible material has an Abbe number of between 25 and 50.
